SEQUENCE LISTING (6 sequences)

SEQ ID NO: 1 - DNA, 181 bp, Artificial Sequence
  Synthetic linker
gcgctgcaggcacctgttgacaattaatcatcggctcgtataatgtgtggaattgtgagcggataacaattatataaggaggtatatcatatgggcccattctagaggatccggtaattaattaatgcataaaacgaaaggctcagtcgaaagactgggcctttcgttttatgaattccgg

SEQ ID NO: 2 - DNA, 3071 bp, Artificial Sequence
  Vector without annexin V gene
ggcacctgttgacaattaatcatcggctcgtataatgtgtggaattgtgagcggataacaattatataaggaggtatatcatatgggcccattctagaggatccggtaattaattaatgcataaaacgaaaggctcagtcgaaagactgggcctttcgttttatgaattcgccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccgtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtga

SEQ ID NO: 3 - DNA, 4044 bp, Artificial Sequence
  Expression vector
ggcacctgttgacaattaatcatcggctcgtataatgtgtggaattgtgagcggataacaattatataaggaggtatatcatatggcgaagtatacaagaggcaccgtgacagcattctctccttttgatgccagagctgatgcagaagcccttcgcaaggccatgaagggaatggggactgatgaagagacaattctgaagatccttaccagcagaaataatgctcaacgtcaagaaattgcatctgcttttaaaacactgtttggcagggatcttgtggatgacctgaaatcagaacttactggcaagtttgaaacactgatggtatctttgatgagaccagcacgtatttttgatgctcatgcactgaagcatgcaatcaagggagcaggaaccaatgagaaagtgttgactgaaattcttgcctccagaacacctgctgaagtgcagaatattaaacaggtttatatgcaagagtatgaggccaacttggaggataagatcacaggagagacatcaggccattttcagagactgctggtggtcctgctgcaggcaaatagagatcctgatggcagagttgacgaggctcttgttgagaaggatgctcaggtcttgtttagagctggggagctaaaatggggaacagatgaagaaacattcatcaccatcttgggaactcgaagtgtttctcatttaaggagggtgtttgacaaatacatgactatttctggctttcaaattgaagaaaccattgaccgtgaaacctctggtgatttggagaagttgcttttggcagttgtgaagtgcatccgaagtgtgcctgcttattttgctgaaactttgtattattccatgaaaggggctggcactgatgatgataccctgatcagagtcatggtttcaagaagtgaaatcgacctgttggatattagacatgaattcagaaagaattttgcgaaatcgttgtatcagatgattcagaaagatacatctggggactacaggaaggccctgctgctcctctgcggcggtgatgacgaataataattaataattaataatctgcaggatccggtaattaattaatgcataaaacgaaaggctcagtcgaaagactgggcctttcgttttatgaattcgccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccgtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtga

SEQ ID NO: 4 - DNA, 5873 bp, Artificial Sequence
  Expression vector
gaattcatggcgaagtatacaagaggcaccgtgacagcattctctccttttgatgccagagctgatgcagaagcccttcgcaaggccatgaagggaatggggactgatgaagagacaattctgaagatccttaccagcagaaataatgctcaacgtcaagaaattgcatctgcttttaaaacactgtttggcagggatcttgtggatgacctgaaatcagaacttactggcaagtttgaaacactgatggtatctttgatgagaccagcacgtatttttgatgctcatgcactgaagcatgctatcaagggagcaggaaccaatgagaaagtgttgactgaaattcttgcctccagaacacctgctgaagtgcagaatattaaacaggtttatatgcaagagtatgaggccaacttggaggataagatcacaggagagacatcaggccattttcagagactgctggtggtcctgctgcaggcaaatagagatcctgatggcagagttgacgaggctcttgttgagaaggatgctcaggtcttgtttagagctggggagctaaaatggggaacagatgaagaaacattcatcaccatcttgggaactcgaagtgtttctcatttgaggagggtgtttgacaaatacatgactatttctggctttcaaattgaagaaaccattgaccgtgaaacctctggtgatttggagaagttgcttttggcagttgtgaagtgcatccgaagtgtgcctgcttattttgctgaaactttgtattattctatgaaaggggctggcactgatgatgataccctgatcagagtcatggtttcaagaagtgaaatcgacctgttggatattagacatgaattcagaaagaattttgcgaaatcgttgtatcagatgattcagaaagatacatctggggactacaggaaggcactcctgctcctctgtggtggagatgatgagtaatggatcaattcccggggatccgtcgacctgcagccaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcagatccgtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacggatctgaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 5 - DNA, 59 bp, Artificial Sequence
  Sense strand of a linker with 2 stop codons in each reading frame
ccctgctgctcctctgcggcggtgatgacgaataataattaataattaataatctgcag

SEQ ID NO: 6 - DNA, 65 bp, Artificial Sequence
  Antisense strand of a linker with 2 stop codons in each reading frame
gatcctgcagattattaattattaattattattcgtcatcaccgccgcagaggagcagcagggcc

(19) United States
(12) Patent Application Publication
(10) Pub. No.: US 2005/0142657 A1
(43) Pub. Date: Jun. 30, 2005

(54) Expression vector for the production of annexin v

(76) Inventors: Swen Grein, Nurnberg (DE); Christian Reiser, Bamberg (DE)

Correspondence Address: FISH & RICHARDSON P.C., PO BOX 1022, MINNEAPOLIS, MN, 55440-1022, US

(21) Appl. No.: 10/499,282

(22) PCT Filed: Dec. 16, 2002
(86) PCT No.: PCT/EP02/14347

(30) Foreign Application Priority Data
Dec. 18, 2001 (DE) 101624344

(51) Int. Cl. C12N 15/00; C12N 15/09; C12N 15/63; C12N 15/70; C12N 15/74

(52) U.S. Cl. 435/320.1

(57) ABSTRACT

The invention relates to an expression vector comprising at least those components of the vectors pBR322 or pKK223-3, necessary for the replication thereof, an expression region sequentially comprising, in the reading direction, a first promotor, a binding site specific for a repressor, a sequence corresponding to a prokaryotic ribosome binding site, a cloning site with a gene coding for annexin V, at least one transcription terminator and at least one kanamycin-resistant gene with a second promotor.

The invention relates to an expression vector and an E. coli cell for production of annexin V.

The expression of eukaryotic genes in bacteria by means of plasmids or vectors as expression vectors is common knowledge. Expression of a VAC protein in E. coli by means of an expression vector, e.g. pBR322, is known from DE 37 10 364 A1. Genes for tetracycline resistance or ampicillin resistance are proposed as antibiotic resistance genes. VAC proteins are also called annexins.

Expression of the placental coagulation inhibitor CPBII, which is also called annexin VI, in E. coli is known from EP 0 351 826 A2. The pKK223-3 expression vector, containing an ampicillin resistance gene, and the E. coli strain JM105 are used for expression.

Expression of annexin V in the E. coli strain BL21 is known from WO 00/73332 A1. Expression vectors have been constructed on the basis of the vector pET12a and contain an ampicillin resistance gene.

Expression of an annexin V gene from the chicken in E. coli by means of the expression vector pTrc99A containing an ampicillin resistance gene is known from Turnay J. et al., Journal of Cellular Biochemistry 58 (1995), pages 208 to 220.

The usual elements of expression vectors are an origin, a gene that imparts an antibiotic resistance, a lac I gene encoding the Lac repressor, a promoter under the control of the lac operator and an optimum ribosome binding site. This is usually followed by a short open reading frame with a multiple cloning site for incorporating a cDNA into the correct reading frame. Such an expression vector is silent in bacteria if the Lac repressor prevents the transcription of the incorporated cDNA. The lac I gene is not required in the vector when the expression vector is used in bacteria in which the lac I gene is expressed in some other way, perhaps because it is present on another vector. By adding the inducer IPTG, transcription can be released in a simple manner. The bacteria then often, but not always, produce the protein encoded by the cDNA. Often the protein is also insoluble and is already precipitated as so-called inclusion bodies in the bacteria. Not every vector with the stated elements is equally suitable for expressing a given protein.

Furthermore, even a protein produced by means of a vector that is very suitable for expression is not necessarily usable as a therapeutic agent. This is because, in particular, the antibiotics usually employed for the selection of successfully transfected cells cannot be separated completely from the protein. The presence of antibiotics in small amounts of the protein administered can lead to the development of resistance to this antibiotic. Furthermore, the antibiotics sometimes have considerable side-effects. Thus, there are often allergies to ampicillin and tetracyclines that are still present in trace amounts in a preparation of the protein. Administration of such a preparation can lead to life-threatening anaphylactic shock. Kanamycin exhibits a high systemic toxicity and therefore should not be administered systemically. Accordingly, even today it is still only used as a therapeutic agent in eye preparations.

The problem of the present invention is to provide an expression vector that is very suitable for the expression of annexin V that can be used systemically as a therapeutic agent. Furthermore, an E. coli cell is to be provided, by means of which annexin V, usable systemically as a therapeutic agent, can be produced.

This problem is solved by the features of claims 1 and 19. Advantageous embodiments can be seen from the features of claims 2 to 18 and 20.

In accordance with the invention, an expression vector is provided that contains the following elements:
at least the components of the vector pBR322 or pKK223-3, which are necessary for its replication
an expression region, which has, following one another in the direction of reading: a first promoter, a binding site specific for a repressor, a sequence corresponding to a prokaryotic ribosome-binding site, a cloning site with a gene coding for annexin V and at least one transcription terminator and
at least one kanamycin resistance gene with a second promoter.

The pBR322 vector is derived from E. coli strain K12 SK1592, which can be obtained from the DSMZ under DSM No. 3879. The pKK223-3 vector is known from Brosius, J. and Holy, A. (1984) Proc. Natl. Acad. Sci. USA 81, pages 6929-6933 and can be obtained for example from the company Amersham Pharmacia Biotech, Freiburg. The “components” are nucleotide sequences that encode the functional units contained in the pBR322 or pKK223-3 vector. These functional units can be genes, for example the rop gene, or nucleotide sequences having some other function, e.g. during replication of the vector. The expression vector can contain all components of the pBR322 or pKK223-3 vector, in particular the complete nucleotide sequence of the vector. However, it is also possible for some components of the vector to be missing, provided they are not required for replication of the vector.

The repressor-specific binding site permits induction of expression of the gene coding for annexin V. The sequence corresponding to the prokaryotic ribosome-binding site is a sequence whose transcription leads to an mRNA sequence representing a prokaryotic ribosome-binding site (Shine-Dalgarno sequence). The prokaryotic ribosome-binding site is necessary for the initiation of translation. The cloning site can be a single cloning site, a multiple cloning site or a cloning site which, through insertion of the gene coding for annexin V, can no longer be digested by restriction enzymes. A multiple cloning site differs from a single cloning site in having more than one restriction site, which permit the incorporation of the various DNAs. At the cloning site, a gene coding for annexin V is contained in the reading frame. For the gene, deviations from the native nucleotide sequence are possible, provided either that they have no effect in the protein sequence or only to an extent that the function of the protein is not lost. The function may also refer to just one of several functions of annexin V. Such functions are for example the ability to bind phosphatidylserine or to inhibit blood coagulation. The expression region without the gene coding for annexin V can be derived, especially in the case of an expression vector containing the components of the pKK223-3 vector, at least partially from the expression region contained in the pKK223-3 vector in the nucleotide sequence 4146 to 72.

Despite the toxicity of kanamycin and the prevailing opinion that its use should therefore be avoided, the expression vector according to the invention has a kanamycin resistance gene. It is especially suitable for the expression of annexin V, usable systemically as a therapeutic agent. Owing to the kanamycin resistance gene, during expression it is unnecessary to use ampicillin or tetracycline, which often cause allergy problems. In an annexin V preparation resulting from expression and purified for therapeutic use, the presence of kanamycin cannot be totally excluded. During purification of annexin V, however, the amount of kanamycin can be reduced to such an extent that its toxicity is no cause for concern at the levels of annexin V usually administered. In contrast, it is difficult to get the levels of ampicillin or tetracycline below the very low concentrations that are sufficient to trigger anaphylactic shock. A further decisive advantage is that, because of its toxicity, kanamycin is no longer employed systemically in therapeutic applications. Consequently the development of kanamycin-resistant pathogens does not represent a substantial risk, otherwise possibly life-threatening when used in an emergency.

Preferably the gene coding for annexin V is derived from a chicken. This gene is particularly suitable for expression by means of the expression vector. The expression vector with the 966-bp annexin V gene from the chicken can have a size of about 4000 base pairs and so is easy to manipulate. To achieve especially efficient expression of annexin V in E. coli, all codons in the gene coding for annexin V can be replaced by codons that are translated preferentially in E. coli. The use of codons that are optimum for E. coli at the 5′ end of an annexin gene is known. This measure is evidently deemed sufficient for achieving increased expression efficiency in E. coli. Surprisingly, a further increase in efficiency can be achieved by replacing all the codons in the annexin V gene by codons that are translated preferentially in E. coli.

Because the stop codon that is present naturally in the annexin V gene can be read through into E. coli, the resulting protein can have additional unwanted amino acids. Therefore preferably at least one additional stop codon is present in the reading frame of the gene, at the 3′ end of the sequence of the gene coding for annexin V. Presence of two additional stop codons is especially preferred. The effect is that the expressed protein ends exactly at the intended point.

The first promoter is preferably a tac promoter. The repressor can be a Lac repressor. The tac promoter corresponds to the tac promoter published by De Boer, H. A. et al. (1983) Proc. Natl. Acad. Sci. USA 80, pages 21-25. It is a hybrid promoter which, in combination with the specific binding site for the Lac repressor, permits expression of the annexin V gene that is inducible e.g. with allolactose or isopropyl-β-D-thiogalactoside (IPTG). Transcription is suppressed in E. coli strains that produce a high level of Lac repressor. These strains are designated as lac I^{q} strains. The transcription terminator can be the transcription terminator rrnB T1 or rrnB T2. It is also possible for the expression vector to contain both rrnB1 T1 and rrnB T2 as transcription terminators. The rrnB T1 and T2 transcription terminators are derived from the rrnB operon in E. coli.

Preferably, at least partially, the expression vector does not contain the ampicillin resistance gene and/or the tetracycline resistance gene, of the components of the pBR322 vector, and the ampicillin resistance gene, of the components of the pKK223-3 vector. This makes it possible for the expression vector to be of small size. Preferably the kanamycin resistance gene contained in the expression vector is derived from the pACYC177 vector or the Tn903 transposon from E. coli. The kanamycin resistance gene from the Tn903 transposon can be obtained as a fragment by digestion of the pUC4K plasmid with the Bam HI restriction endonuclease. The pUC4K plasmid can be obtained, for example, from the company Amersham Pharmacia Biotech, Freiburg. The pACYC177 vector is derived from E. coli strain PC2166, which can be obtained from the Deutsche Sammlung von Mikroorganismen und Zellkulturen, Braunschweig (DSMZ), under DSM No. 5587.

The second promoter is preferably the pK promoter from the pACYC177 vector. This promoter is strong enough to ensure that expression of the kanamycin resistance gene is sufficiently high for kanamycin resistance. At the same time the pK promoter does not cause expression that is so strong that the expression of annexin V is substantially reduced as a result. The kanamycin resistance gene with the second promoter can be inserted in the expression vector at the Eco RI and Sty I restriction sites derived from the pBR322 vector or the Eco RI restriction site derived from the pKK223-3 vector. Preferably the kanamycin resistance gene with the second promoter pK in the nucleotide sequence 1816 to 2771 of the pACYC177 vector is contained in the expression vector.

The expression region can be inserted in the expression vector at the Ban I and Eco RI restriction sites derived from the pBR322 vector. Preferably the expression region without the gene coding for annexin V has the sequence SEQ ID NO: 1 according to the appended sequence listing.

In one embodiment, no cleavage site located outside of the cloning site, for a restriction endonuclease of class II, which has a recognition sequence with the nucleotide sequence ATG, is contained in the expression vector. Restriction endonucleases of class II recognize a specific nucleotide sequence and cleave specifically in this sequence or near this sequence. A restriction endonuclease of class II, which has a recognition sequence with the nucleotide sequence ATG, permits simple cloning of the ATG start codon of a gene that is to be expressed. In prokaryotes, the optimum distance from the ribosome-binding site, ensuring efficient translation of the mRNA, is 4 to 8 nucleotides between the last nucleotide of the ribosome-binding site and the A of the start codon. If there is no cleavage site for the stated restriction endonuclease outside of the cloning site, it is possible to use this cleavage site specifically in the cloning site, to permit simple cloning of the start codon of the annexin V gene to be expressed, at an optimum distance from the ribosome-binding site. The restriction sites can be removed by directed mutagenesis. The restriction endonucleases Nde I and Nco I are preferred. The recognition sequence of both restriction endonucleases contains the nucleotide sequence ATG. Otherwise, however, the recognition sequences seldom occur, so that only a few cleavage sites outside of the cloning site have to be removed. A cleavage site requiring removal for the restriction endonuclease Nde I can originate from the pBR322 vector.

The expression vector without the gene coding for annexin V can have the sequence SEQ ID NO: 2 according to the appended sequence listing. The expression vector with components of the pBR322 vector and an annexin V gene derived by codon optimization from the annexin V gene from the chicken can have the sequence SEQ ID NO: 3 according to the appended sequence listing. This sequence contains two additional stop codons in the reading frame for annexin V. The expression vector with components of the pKK223-3 vector and the annexin V gene of the chicken can have the sequence SEQ ID NO: 4 according to the appended sequence listing.

A further object of the invention relates to an E. coli cell that contains an expression vector according to the invention. Preferably the E. coli cell belongs to the strain BL21. This strain can be obtained for example from the company Novagen, Inc., 601 Science Drive, Madison, Wis. 53711, USA. This cell has proved to be especially suitable for the expression of annexin V. The E. coli cell can also belong to a lac I^{q} strain, especially the strain JM105. An E. coli cell of a lac I^{q} strain is especially suitable for the expression of annexin V in an expression vector in which the first promoter is the tac promoter. E. coli cells of the strain JM105 can be obtained for example from the American Type Culture Collection (ATCC), Manassas, Va. 20108, USA.

An expression vector according to the invention for the expression of annexin V can be produced by a method with the following steps:
a1) production of a first vector by complete or partial removal of the components of the pBR322 vector not required for replication or by PCR amplification of at least the components of the pBR322 vector that are required for replication of the vector, followed by ligation,
   synthesis of an expression region, which has, following one another in the direction of reading: a first promoter, a repressor-specific binding site, a sequence corresponding to a prokaryotic ribosome-binding site, a cloning site and a transcription terminator and insertion of the expression region into the pBR322 vector, the first vector or a vector derived from this vector by one of the steps of this method or
a2) production of a first vector by complete or partial removal of the components not required for replication with the exception of an expression region of the pKK223-3 vector having a cloning site or by PCR amplification of the expression region having a cloning site and at least the components of the pKK223-3 vector that are required for replication of the vector, followed by ligation and
b) insertion of at least one functionally active kanamycin resistance gene in the pBR322 vector, the pKK223-3 vector, the first vector or a vector derived from these vectors by one of the steps of this method, if otherwise no functionally active kanamycin resistance gene is present in the first vector and
c) insertion of a gene coding for annexin V into the cloning site.

Insertion and removal are generally effected by a usual cloning step. It is to be understood that steps b) and c) are carried out both in a method with step A1) and in a method with step A2). In the case of step A1), removal or PCR amplification can take place on the pBR322 vector or a vector produced by the method, in particular by insertion of the expression region. An expression region that is already present in the vector in steps A1) and A2) is amplified during the PCR amplification in connection with the components of the vector. Ligation is necessary in order for the free ends that arose during PCR amplification to be closed to a vector. During this, the free ends of the vector can be ligated together or with free ends of a DNA that is to be inserted, especially of the expression region or of the gene coding for annexin V. The kanamycin resistance gene is functionally active if, after insertion, a second promoter makes expression of the kanamycin resistance gene possible. The functionally active kanamycin resistance gene includes the second promoter, if it is not present in the vector.

The gene coding for annexin V can be derived from a chicken. Preferably, the codons in the gene coding for annexin V are replaced by codons that are translated preferentially in E. coli. This codon optimization can be achieved by directed mutagenesis or de novo synthesis. In one embodiment of the method, at least one additional stop codon in the reading frame of the gene is inserted at the 3′ end of the sequence of the gene coding for annexin V. The insertion of two additional stop codons is especially advantageous. Preferably the stop codon is inserted by means of a linker, in particular having the sense-strand sequence SEQ ID NO: 5 and the antisense-strand sequence SEQ ID NO: 6 according to the appended sequence listing. Insertion is effected preferably into a cDNA coding for annexin V prior to insertion into the cloning site, insertion also meaning terminal attachment. Attachment of the linker with the sequences SEQ ID NO: 5 and SEQ ID NO: 6 according to the appended sequence listing is preferably effected via a Bse RI restriction site present in the annexin V cDNA.

The first promoter is preferably a tac promoter. The repressor can be a Lac repressor. The transcription terminator is preferably the transcription terminator rrnB T1 or rrnB T2 derived from E. coli. It is also possible for both rrnB1 T1 and rrnB T2 to be transcription terminators. Preferably, during production of the first vector, the ampicillin resistance gene and/or at least one part of the tetracycline resistance gene is removed or not amplified by PCR. Prior to insertion, the kanamycin resistance gene can be amplified by a PCR or can be isolated from a plasmid. Insertion of the second promoter is not necessary if the vector contains a promoter that makes expression of the kanamycin resistance gene possible. Preferably the kanamycin resistance gene is derived from the pACYC177 vector or the transposon Tn903 derived from E. coli. The kanamycin resistance gene can be inserted with a second promoter, especially the pK promoter from the pACYC177 vector. The kanamycin resistance gene with the second promoter is preferably inserted at the restriction sites Eco RI and Sty I derived from the pBR322 vector, or the restriction site Eco RI derived from the pKK223-3 vector. This also comprises insertion into the pBR322 or pKK223-3 vector itself. In one embodiment, the kanamycin resistance gene with the second pK promoter is obtained by PCR amplification of the nucleotide sequence 1816 to 2771 of the pACYC177 vector. The expression region is preferably inserted at the restriction sites Ban I and Eco RI derived from the pBR322 vector. In this case insertion can also be into the pBR322 vector itself. In one embodiment, the expression region without the gene coding for annexin V has the sequence SEQ ID NO: 1 according to the appended sequence listing.

It is especially advantageous if a cleavage site, located outside of the cloning site, for a restriction endonuclease of class II, which has a recognition sequence with the nucleotide sequence ATG, is removed or is altered in such a way, in particular by directed mutagenesis, that the restriction endonuclease can no longer effect cleavage at the cleavage site. In order for the advantages of an ATG sequence explained above to be utilized in the recognition sequence, removal or alteration must take place prior to insertion of a gene that is to be expressed. The restriction endonuclease is preferably Nde I or Nco I. In one embodiment the expression vector without the gene coding for annexin V has the sequence SEQ ID NO: 2 according to the appended sequence listing. The complete expression vector can have the sequence SEQ ID NO: 3 according to the appended sequence listing. This sequence contains a gene derived by codon optimization from the annexin V gene from the chicken. Two additional stop codons in the reading frame for annexin V have been inserted in this sequence. The expression vector can also have the sequence SEQ ID NO: 4 according to the appended sequence listing.

The invention is explained below on the basis of possible cloning schemes. In the drawings:

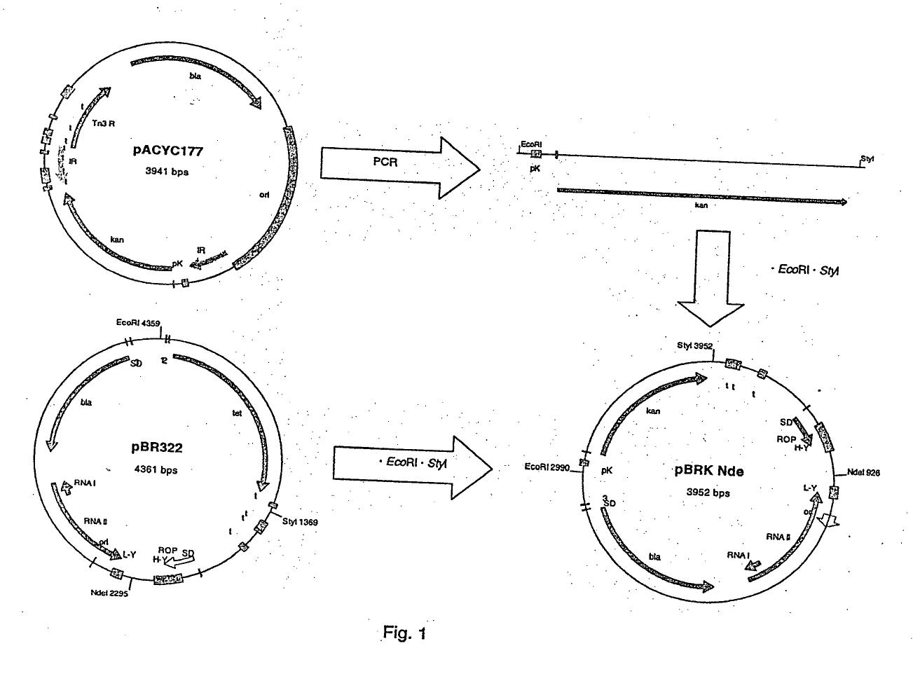

FIG. 1 shows insertion of the product of PCR amplification of the kanamycin resistance gene with the associated promoter pK from the pACYC177 vector in the pBR322 vector,

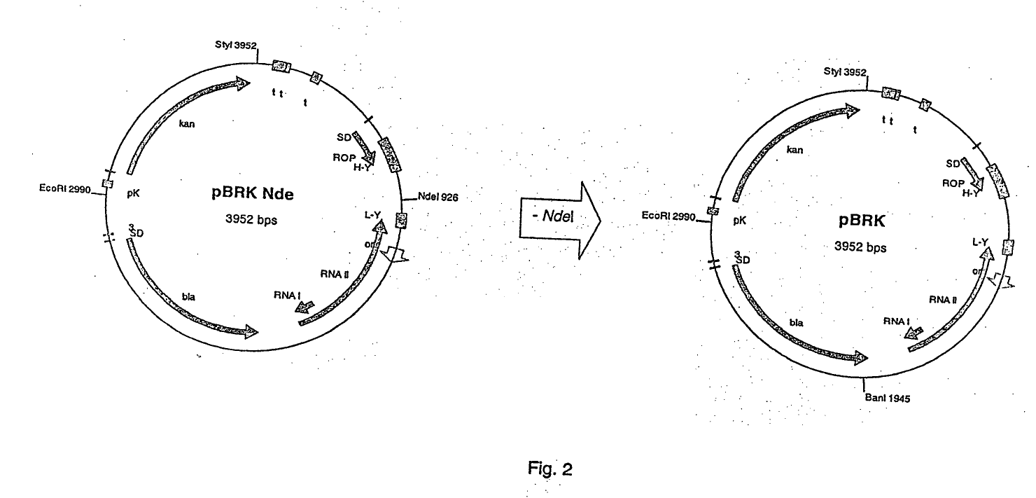

FIG. 2 shows removal of the Nde I restriction site in the vector resulting therefrom and

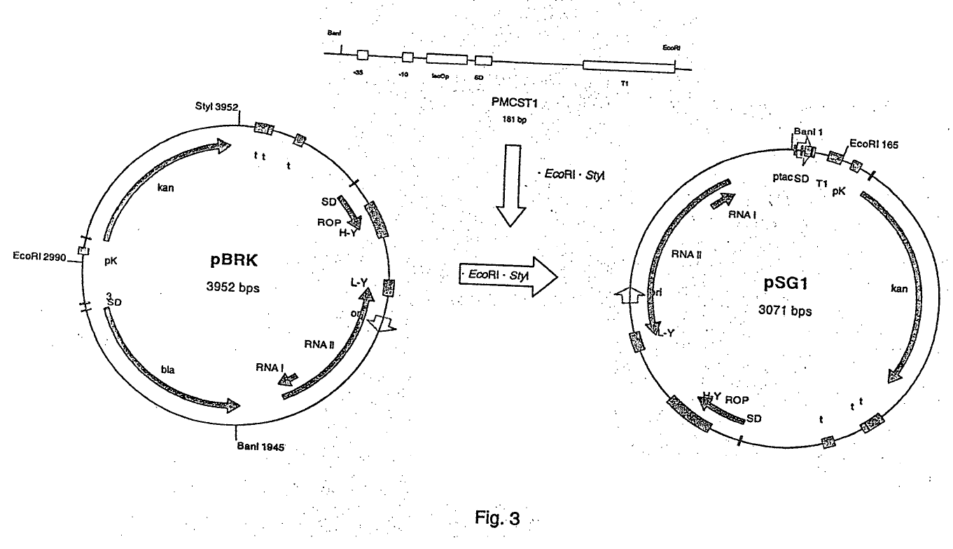

FIG. 3 shows insertion of the expression region into the resultant vector.

From the pACYC177 vector shown in FIG. 1, the nucleotides 1816-2771 comprising the kanamycin resistance gene with the associated promoter pK are amplified by PCR. The product of this reaction is digested with the Eco RI and Sty I restriction enzymes. It is cloned into the pBR322 vector, which was also digested with these enzymes.

The Nde I restriction site is removed from the resultant vector by directed mutagenesis. This is shown schematically in FIG. 2. Cloning of the synthetically produced expression region into the resultant vector at the restriction sites Ban I and Eco RI is shown schematically in FIG. 3.

Poly A⁺ RNA from chicken liver is reverse-transcribed for production of whole cDNA. cDNA coding for annexin V is amplified by PCR from the whole cDNA. The amplified cDNA is digested enzymatically at the restriction sites introduced during PCR by corresponding primers for the restriction endonucleases Nde I and Bam HI. This cDNA is cloned into the vector at the restriction sites Bam HI and Nde I that are also present in the vector in the region between the ribosome-binding site labeled SD and the transcription terminator labeled T1.

In a further cloning scheme, the entire sequence of the pKK223-3 vector apart from the gene coding for ampicillin resistance and the associated promoter is amplified by a PCR. The primers used for this have the sequences SEQ ID NO: 6 and SEQ ID NO: 7 according to the appended sequence listing. The amplified sequence is cleaved at the cleavage sites introduced by each of the primers for the restriction enzyme Bgl II. The kanamycin resistance gene derived from the Tn903 transposon from E. coli is obtained as a fragment of the pUC4K vector by digestion of this vector with the Bam HI restriction endonuclease. The fragment displaying kanamycin resistance and the amplified sequence are ligated at the compatible cohesive ends of the cleavage sites that arose by digestion with Bam HI and Bgl II to a first vector. The cloning site it contains, in the expression region derived from the pKK223-3 vector, is cleaved with the Eco RI restriction endonuclease. The resultant nucleotide overhangs are filled-in by means of the Klenow fragment of DNA polymerase I and are dephosphorylated with alkaline phosphatase.

Poly A⁺ RNA is isolated from embryonic chicken fibroblasts. cDNA is obtained from it by reverse transcription. cDNA coding for annexin V is amplified from that by PCR. The amplified cDNA is digested with the Nco I restriction endonuclease and then with the Bam HI restriction endonuclease. The nucleotide overhangs of the fragment coding for annexin V are filled-in by means of the Klenow fragment of DNA polymerase I. The resultant fragment is ligated with the Eco RI-cleaved filled-in and desphosphorylated first vector to an expression vector according to the invention.

1-20. (canceled)

21. An expression vector comprising:
at least one of the components of the pBR322 or pKK223-3 vector required for replication;
an expression region that has, following one another in the direction of reading: a first promoter, a repressor-specific binding site, a sequence corresponding to a prokaryotic ribosome-binding site, a cloning site with a gene coding for annexin V, and at least one transcription terminator; and
at least one kanamycin resistance gene with a second promoter.

22. The expression vector according to claim 21, wherein the gene coding for annexin V is derived from a chicken.

23. The expression vector according to claim 21, wherein all the codons in the gene coding for annexin V have been replaced by codons that are preferential for E. coli.

24. The expression vector according to claim 21, wherein at least one additional stop codon is present in the reading frame, at the 3′ end of the sequence of the gene coding for annexin V.

25. The expression vector according to claim 21, wherein the first promoter is a tac promoter.

26. The expression vector according to claim 21, wherein the repressor-specific binding site binds a Lac repressor.

27. The expression vector according to claim 21, wherein the transcription terminator is rrnB T1 or rrnB T2.

28. The expression vector according to claim 21, wherein the expression vector does not contain the entire ampicillin resistance gene from the pBR322 vector or the pKK22303 vector.

29. The expression vector according to claim 21, wherein the expression vector does not contain the entire tetracycline resistance gene from the pBR322 vector.

30. The expression vector according to claim 21, wherein the kanamycin resistance gene is derived from the pACYC177 vector or the Tn903 transposon from E. coli.

31. The expression vector according to claim 21, wherein the second promoter is the pK promoter from the pACYC177 vector.

32. The expression vector according to claim 21, wherein the kanamycin resistance gene with the second promoter is inserted into the expression vector at the EcoRI and StyI restriction sites derived from the pBR322 vector or the EcoRI restriction site derived from the pKK223-3 vector.

33. The expression vector according to claim 21, wherein the kanamycin resistance gene with the second promoter corresponds to the nucleotides 1816 to 2771 of the pACYC177 vector.

34. The expression vector according to claim 21, wherein the expression region is inserted into the expression vector at the BanI and EcoRI restriction sites derived from the pBR322 vector.

35. The expression vector according to claim 21, wherein the expression region without the gene coding for annexin V has the sequence shown in SEQ ID NO: 1.

36. The expression vector according to claim 21, wherein the expression vector does not contain a class II restriction endonuclease site outside of the cloning site that has an ATG in the recognition sequence.

37. The expression vector according to claim 36, wherein the class II restriction endonuclease is NdeI or NcoI.

38. The expression vector according to claim 21, characterized in that the expression vector without the gene coding for annexin V has the sequence shown in SEQ ID NO: 2.

39. The expression vector according to claim 21, characterized in that the expression vector has the sequence shown in SEQ ID NO:3 or SEQ ID NO:4.

40. An E. coli cell containing an expression vector according to claim 21.

41. The E. coli cell according to claim 40, characterized in that the E. coli cell belongs to a BL21 strain or a lac I^{q} strain.

42. The E. Coli cell according to claim 41, wherein the lac I^{q} strain is a JM105 strain.
